(19) United States
(12) Patent Application Publication
(10) Pub. No.: US 2005/0066463 A1
(43) Pub. Date: Mar. 31, 2005

(54) Substrates and devices for applying a lip care formulation

(76) Inventor: Michael S. Brunner, Roswell, GA (US)

Correspondence Address: KIMBERLY-CLARK WORLDWIDE, INC., 401 NORTH LAKE STREET, NEENAH, WI, 54956, US

(21) Appl. No.: 10/671,388

(22) Filed: Sep. 25, 2003

(51) Int. Cl. A61K 7/025; A46B 9/04

(52) U.S. Cl. 15/167.2

(57) ABSTRACT

A disposable device that includes a fabric substrate on which a lip care formulation is included is provided.

### TECHNICAL FIELD

This invention relates to substrates and devices for applying a lip care formulation.

### BACKGROUND

People use lip care formulations for one or more of a variety of reasons, for example: to treat and condition dry lips, to moisturize lips, to treat cold sores and herpes sores, to prevent drying and cracking and to maintain overall lip health. Typically, lip care formulations are provided in a stick or in tube and the consumer contacts the composition each time risking contamination from previous use by the consumer or by another previous consumer. Thus, the consumer could come into contact with infectious microorganisms from a previous use. Accordingly, there is a need for a device for applying a lip care formulation that reduces the risk of contamination.

In addition, new oral cleaning devices have recently been developed as that are designed to fit onto the finger of a user so that the user can then insert the device covered finger into the user's mouth, an infant's mouth or an animal's mouth to clean the teeth of the user, an infant, a child, an elderly or incapacitated person or even pet. Exemplary oral cleaning devices are described and illustrated in U.S. Patent Application Publication no. 2002/0170133 A1. Advantageously, these devices can be used during the day as an alternative to toothbrushes. The dental wipes fit onto a human finger so that the teeth or gums of a person or animal can be cleaned by simply contacting the wipe therewith. In addition, the dental wipe is particularly well suited for use by small children learning how to clean their teeth. However, frequent use of such oral care devices could remove moisture from lips potentially drying the lips and reducing the barrier properties of the lips to elements such as dry air, heat cold, and germs. Accordingly, there is a need for a dental wipe that can be used to clean teeth or other mouth surface and that also provides a lip care formulation to provide additional benefits.

### BRIEF DESCRIPTION OF THE INVENTION

Single-use devices for providing and applying a lip care formulation are described. In one embodiment, the device is a hollow member having an open end for insertion of a finger that includes an exterior surface and a lip care formulation disposed on at least a portion of the exterior surface. The device may further include a texturized surface configured to clean the teeth and gums. The portion of the surface of the device that includes the lip care formulation can be separate from the texturized surface or a portion of the texturized surface. Alternatively, the portion of the surface that includes the lip care formulation can be a portion of the texturized surface. In one embodiment, the portion of the surface that includes the lip care formulation is closer to the open end of the device than the texturized surface. The device may include an optional breath freshening agent, flavoring agent or dental agent. The breath freshening agent, the flavoring agent or a dental agent such as a fluoride can be included on a portion of the surface of the device that is separate from the portion of the surface of the device that includes the lip care formulation. The lip care formulation may include one or more of the following compounds: allantoin, avobenzone, camphor, dimethicone, homosalate, menthol, meradimate, octinoxate, octisalate, oxybenzone, padimate O, petrolatum, and phenol. For example, the lip care formulation may include at least one sun screening agent, at least one sun screening agent or at least one a skin protecting agent or a combination thereof. In one embodiment, the lip care formulation comprises an analgesic and a skin protecting agent. The device analgesic may include camphor, menthol and phenol. The device skin protecting agent may include allantoin, dimethicone and petrolatum. And, the sun screening agent may include aminobenzoic acid, anthranilate avobenzone, dioxybenzone, homosalate, lisadimate, menthyl anthranilate, meridamate, octinoxate, octisalate, octocrylene, octyl methoxycinnamate, octyl salicylate, oxybenzone, padimate O, phenylbenzimidazole, roxadimate, sulisobenzone, titanium dioxide, trolamine salicylate or zinc oxide.

In another embodiment, the present invention is a disposable substrate for applying a lip treatment composition that is or includes a nonwoven material having a surface upon which is disposed a lip care formulation. Devices of the present invention may be packaged to provide a packaged disposable product for applying a lip treatment composition that includes a disposable nonwoven substrate upon which is disposed a lip care formulation.

### BRIEF DESCRIPTION OF THE DRAWINGS

The invention will be more fully understood and further advantages will become apparent when reference is made to various embodiments described in the following description and the accompanying drawings in which:

FIG. 1 illustrates a perspective view of a wipe according to one embodiment of the present invention;

FIG. 2 illustrates a perspective view of a dental wipe on a finger according to another embodiment of the present invention;

FIG. 3 is a perspective view of a two-sided dental wipe according to yet another embodiment of the present invention;

FIG. 4 is a perspective view of a bottom section of a two-sided dental wipe according to one embodiment of the present invention;

FIG. 5 is a perspective view of the two-sided dental wipe of FIG. 4.

### DETAILED DESCRIPTION

Reference now will be made to the embodiments of the invention, one or more examples of which are set forth below. Each example is provided by way of explanation of the invention, not as a limitation of the invention. In fact, it will be apparent to those skilled in the art that various modifications and variations can be made in this invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention, which broader aspects are embodied in the exemplary constructions.

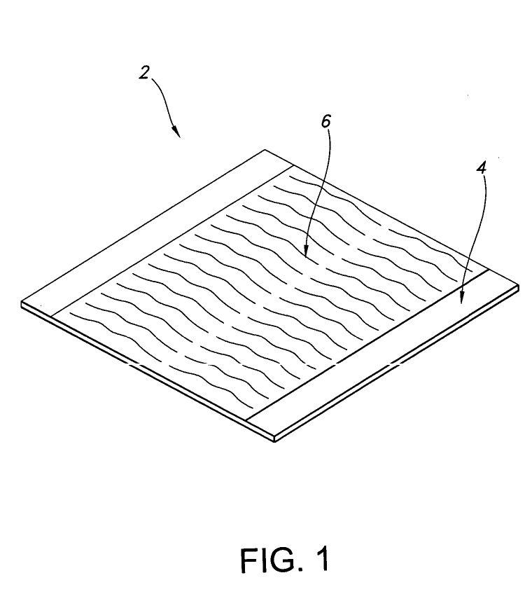

In general, the present invention is directed to a disposable device for applying a lip care formulation. The lip care formulation can be any compound, composition or formulation that includes a compound that is or can be used to protect, repair, moisturize or otherwise provide relief to damaged or undamaged skin, particularly lips. The device can be a substrate, for example a small piece of fabric such as a disposable nonwoven fabric upon which a lip care formulation has been applied or a more complicated device. In certain embodiments, the device is as a disposable wipe that includes a portion to which a lip care formulation has been applied. One example of this embodiment is illustrated in FIG. 1. FIG. 1 illustrates a disposable nonwoven substrate 2, one surface 4 of which is coated with a lip care formulation 6. All or a portion of the surface 4 can be coated with the lip care formulation 6. The lip care formulation 6 or the surface 4 can be colored so that a consumer will have a visual indication as to which side of the substrate 2 is provided with a transferable lip care formulation 6. The wipes can be individually packaged.

In other embodiments the present invention includes dental wipes that further provide a lip care formulation. Various exemplary and suggested dental wipes and related devices for cleaning teeth are described in International Patent Publication No. WO 01/76521 and U.S. Patent Application Publication no. 2002/0170133 A1 to McDevitt et al. which are hereby incorporated by reference in their entireties herein. Thus, in one embodiment the present invention provides a disposable device for applying a lip care formulation that is a dental wipe. A dental wipe of the present invention can be used to apply a lip care formulation as well as to clean teeth.

The present invention will now be described with reference to this particular embodiment. Substrates, devices and dental wipes made in accordance to the present invention are generally constructed from disposable materials, such as nonwoven webs made from synthetic and/or pulp fibers. In one embodiment, a dental wipe of the present invention typically includes a texturized surface adapted to scrub or clean the teeth and/or gums of a user and a portion of the dental wipe, for example a portion of the texturized surface, is provided with a lip care formulation. The dental wipe can also include an elastic component for providing the wipe with form-fitting properties. By forming a dental wipe with an elastic component, the resulting wipe can snugly fit onto a person's finger so that the wipe can more effectively remain on the finger throughout the cleaning process. Moreover, a dental wipe of the present invention can remain “breathable” to aid in a person's comfort during use, while also remaining capable of substantially inhibiting the transfer of liquids from the outer surface of the wipe to the person's finger. The transfer of liquids can be controlled using a liquid-impervious material and/or by using a highly liquid absorbent material.

A dental wipe of the present invention can generally be formed in a variety of ways as described in detail in U.S. Patent Application Publication no. 2002/0170133 A1 to McDevitt et al. For instance, in one embodiment, the dental wipe can be formed as a unitary structure from a particular base web material, such as an elastomeric nonwoven base web material. Moreover, in another embodiment of the present invention, the dental wipe can be formed from two or more sections of base web material. Each section can be identical or different, depending on the desired characteristics of the dental wipe. For example, in one embodiment, the dental wipe is formed from two sections, wherein one section is formed from a texturized nonwoven material and the other section is formed from an elastomeric nonwoven material.

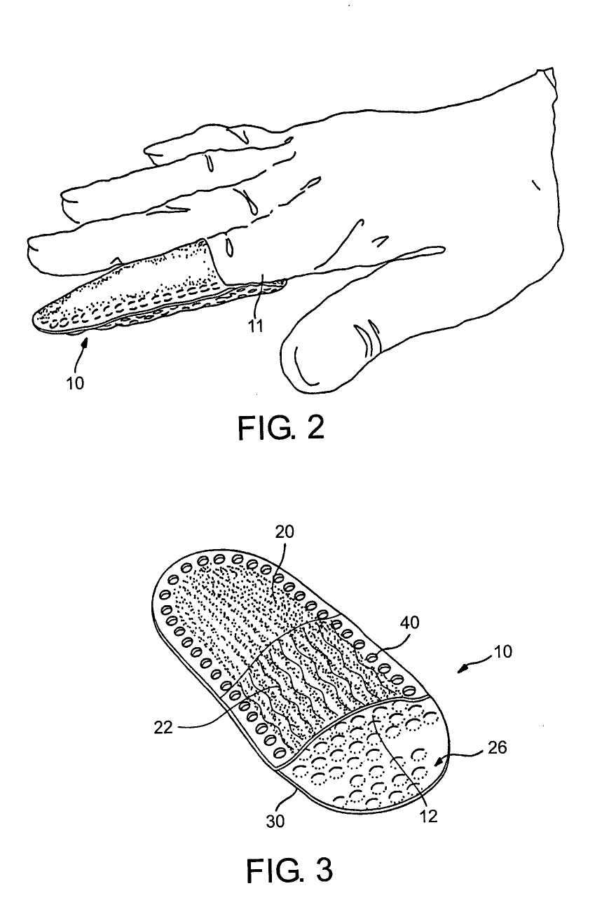

Referring to FIGS. 2-5, various embodiments of dental wipes made in accordance with the present invention are depicted. In general, a dental wipe of the present invention can be used to clean the oral cavity of a user by inserting the wipe onto a finger and maneuvering it within the oral cavity and then can be used to apply a lip care formulation that is provided on one or more surfaces of the wipe to the user's lips by contacting the portion including the formulation to the user's lips. In particular, as shown in FIG. 2, a dental wipe 10 can be placed over a finger 11 for cleaning, then inserted into a mouth for cleaning teeth and/or gum surfaces, withdrawn and then a portion of the wipe can be contacted to one or both lips to transfer a lip care formulation to one or both lips. For example, the wipe portion that includes a lip care formulation can be contacted to one lip to transfer and apply lip care formulation to that lip and then the user can contact one lip to the other lip to transfer and apply lip care formulation to both lips. In certain embodiments, the wipe can be simply a piece of disposable fabric upon which a lip care formulation is included or can be formed as a unitary structure from a single piece of fabric as illustrated in FIGS. 8 and 9 of U.S. Patent Application Publication no. 2002/0170133 A1 to McDevitt et al. upon which a transferable lip care formulation has been included.

FIGS. 2-5 illustrate an embodiment of the present invention in which the device is a dental wipe 10 that is made from a first section 20 and a second section 30. Generally, one section of the dental wipe 10 can be bonded or attached to the other section in a finger-shaped pattern by any manner known in the art, such as by adhesive, thermal, or mechanical bonding, so that the connection of the sections can form a pocket for the insertion of a finger, as shown in FIG. 2. In the embodiment depicted in FIG. 3, for example, the first section 20 is attached in a finger-shaped pattern to the second section 30 at their respective outer edges via the seams 40 to form a dental wipe 10 having a pocket 12. Once each section is bonded or attached at the seams 40, the materials forming each of the sections 20 and 30 can then be cut adjacent to the seams such that the finger-shaped dental wipe 10 is formed. A lip care formulation can be applied to an exterior portion of the wipe 22 before or after the wipe is formed. In FIG. 3, a lip care formulation is provided on an exterior portion of the wipe 22 that is an upper surface of the wipe that is proximate the open end of the illustrated dental wipe.

As shown in FIGS. 2-5, the first section 20 can also, in some embodiments, have a length greater than the second section 30 such that the first section 20 includes a portion (or pull-on tab) 26 that extends beyond the edge of the second section 30. By extending beyond the second section 30, the portion 26 can be used as a pull-on tab to facilitate placement of the dental wipe 10 over a finger. In particular, a user can conveniently grab the portion 26 to place the dental wipe 10 over a finger. Besides the first section 20, a pull-on tab 26 can be positioned on any suitable portion of the dental wipe. For instance, the pull-on tab 26 can be located on the second section also. Further, in another embodiment, the pull-on tab 26 can also be provided in the middle portion of the dental wipe 10 such that a user can pull the pull-on tab 26 in a direction perpendicular to the lengthwise direction of a flattened dental wipe as shown in phantom in FIG. 5. As a result, the pull-on tab 26 can facilitate the insertion of a finger into the wipe 10 by “spreading out” the sleeve in an upwardly direction as a finger is inserted therein. The first section 20 includes a central region 22a that is intermediate the closed end and the open end of the wipe and a basal region 22b that hat is proximate the closed end of the wipe, The second section 30 includes a central portion 32 and a contacting region 33. The lip care formulation can be included on any exterior portion or section of the wipe 10 that is amenable for applying and transferring the formulation. For example, suggested locations for applying or otherwise including a lip care formulation include, but are not limited to, a central region 22a of the upper surface that is in intermediate the closed end and the open end of the wipe, a basal region 22b of the upper surface that is proximate the closed end of the wipe, and a central portion 32 of the lower surface of the wipe. A lip care formulation can be applied to any one of these areas, a combination of these areas or on any other portion of the device that is amenable for applying and transferring a lip care formulation to lips. The lip care formulation may include an optional colorant or a die to provide a visual indication of a portion of the wipe that includes the lip care formulation to a consumer. Alternatively, the portion of the wipe that is used to clean teeth and/or gums can be colored differently.

Moreover, although not specifically shown, a dental wipe of the present invention can include bristles or loops on the first section 20 and/or the second section 30. For example, bristles such as described in U.S. Pat. No. 4,617,694 to Bori or U.S. Pat. No. 5,287,584 to Skinner, which are incorporated herein by reference, can be utilized with a dental wipe of the present invention. The lip care formulation may be provided on the portion that includes bristles, on a portion that does not include bristles or a portion that overlaps a portion including bristles and an adjacent portion of the device that does not include bristles. Further, a dental wipe 10 can also be provided with a tapered shape to enhance the ability of the wipe to fit onto a finger. In addition, a dental wipe 10 can have two open as described and illustrated in greater detail in U.S. Patent Application Publication no. 2002/0170133 A1 to McDevitt et al. so that a finger can be inserted completely therethrough.

Lip Care Formulation

As previously stated the lip care formulation can be any compound or any composition or formulation that includes a compound that is or can be used to protect, repair, moisturize or otherwise provide relief to damaged or undamaged skin, particularly lips. Lip care formulations may include one or more of the following active ingredients: an analgesic to provide pain relief, a moisturizing agent to maintain or improve skin dryness, a skin protecting agent to protect the skin from the environment, and a sun screening agent to protect the skin from the sun. Analgesics are known and include any compound, composition or formulation that includes a compound that eliminates or reduces pain. Suggested analgesic compounds for lip care formulations include, but are not limited to, salicylic acid, alum salicylic acid, camphor, menthol, phenol and their derivatives and so forth. Commercially available analgesic formulations include, but are not limited to, BLISTEX® Lip Ointment and BLISTEX® Lip MEDEX® both of which are available from Blistex Inc, of Oakbrook, Ill.; CARMEX® For-Cold-Sores which is available from Carma Laboratories, Inc. of Franklin, Wis. The lip care formulation may be or may include any of these commercially available formulations or may include any of the active ingredients contained therein or any other compound that can be used to can be used to protect, repair, moisturize or otherwise provide relief to damaged or undamaged lips. moisturizing agents include, but are not limited to, N-acetyl ethanolamine, aloe vera gel, arginine PCA, chitosan PCA, copper PCA, corn glycerides, dimethyl imidazolidinone, fructose, glucamine, glucose, glucose glutamate, glucuronic acid, glutamic acid, glycereth-7, glycereth-12, glycereth-20, glycereth-26, glycerin, honey, hydrogenated honey, hydrogenated starch hydrolysates, hydrolyzed corn starch, lactamide MEA, lactic acid, lactose lysine PCA, mannitol, methyl gluceth-10, methyl gluceth-20, PCA, PEG-2 lactamide, PEG-10 propylene glycol, polyamino acids, polysaccharides, polyamino sugar condensate, potassium PCA, propylene glycol, propylene glycol citrate, saccharide hydrolysate, saccharide isomerate, sodium aspartate, sodium lactate, sodium PCA, sorbitol, TEA-lactate, TEA-PCA, urea, xylitol, panthenol, petrolatum, mineral oil, lanolin, lanolin alcohol, tocopherol, esters of tocopherol, alkyl polydimethylsiloxanes, vegetable oils, hydrogenated vegetable oils, fatty acid esters, beeswax and so forth. Suggested moisturizing agents include glycerin and petrolatum and mixtures thereof. Suggested skin protecting agents include, but are not limited to, allantoin, dimethicone petrolatum and so forth. The lip care formulation may include one or more sunscreen agents. Sun screening agents protect skin from harmful effects of the sun, for example ultraviolet radiation. Sun screening agents are known. Desirably, the sun screening should be safe for human use and approved for human use. Suggested sun screening agents include, but are not limited to, aminobenzoic acid, anthranilate avobenzone, dioxybenzone, homosalate, lisadimate, menthyl anthranilate, meridamate, octinoxate, octisalate, octocrylene, octyl methoxycinnamate, octyl salicylate, oxybenzone, padimate O, phenylbenzimidazole, roxadimate, sulisobenzone, titanium dioxide, trolamine salicylate, zinc oxide and so forth.

The lip care formulation may further include one or more of the following optional ingredients: botanical extracts, moisturizing agents, skin protecting agents, sun screen agents, dyes, colorants and so forth. Suggested botanical extracts include, but are not limited to, aloe barbadensis leaf extract, aloe extract, avocado oil, calendula officinalis flower extract, camphor, castor oil, chamomile extract, chamomilla recutita (matricaria) flower extract, green tea extract, daucus carota sativa (carrot root), helianthus annuus (sunflower) seed oil, persea gratissima (avocado) oil, jojoba esters, jojoba oil, krameria triandra root extract, ricinus communis (castor) seed oil, sesamum indicum (sesame) seed oil, simmondsia chinensis (jojoba) seed oil, and so forth.

The lip care formulation may include one or more other optional ingredients such as solidifiers and binders. Suggested optional ingredients include, but are not limited to, waxes, ammonium hydroxide, alumina, aluminum hydroxide, arachidyl alcohol, beeswax, behenoyl stearic acid, behenyl alcohol, benzoic acid, benzyl alcohol, brassica campestris/aleurites fordi oil copolymer, butylparaben, butyrospermum parkii (shea butter) fruit, calcium disodium EDTA, calcium hydroxide, candelilla wax, caprylic/capric triglyceride, carvone, cetyl alcohol, cetyl dimethicone, cetyl palmitate, cholesteryl/behenyl/octyldodecyl lauroyl glutamate, cocoa butter, cocoyl hydrolyzed soy protein, cyclomethicone, diethylhexyl adipate, diethylhexyl naphthalate, diisopropyl adipate, diisostearate, dimethicone, dioctyl adipate, ethylhexyl palmitate, ethylhexyl stearate, ethylparaben, euphorbia cerifera (candelilia) wax, glycerin, glyceryl laurate, glyceryl state, glycine, hydrogenated castor oil, hydrogenated coco-glycerides, hydrogenated polyisobutane, hydrogenated soybean oil, hydrogenated vegetable oil, hydroxylated milk glycerides, hydroxystearic acid, iron oxide, isobutylparaben, isohexadecane, isopropyl lanolate, isopropyl myristate, isopropyl palmitate, isopropyl stearate, isostearoyl hydrolyzed silk, isostearyl alcohol, lanolin, lanolin oil, lauric acid, lecithin, menthol, menthoxypropanediol, meradimate, methylparaben, mica, microcrystalline wax, mineral oil, myristic acid, myristyl myristate, octyl palmitate, octyl stearate, octyidodecyl neopentanoate, oleic acid, oryzanol, ozokerite, palmitic acid, panthenol, panthenyl ethyl ether, pantothenic acid polypeptide, paraffin, pentaerythrityl tetraoctanoate, petrolatum, phenol, phenoxyethanol, phenyl trimethicone, polybutene, polyethylene, polyhydroxystearic acid, potassium hydroxide, propylparaben, purified water, PVP/eicosene copolymer, red, retinyl palmitate, retinyl palmitate polypeptide, saccharin, SD alcohol 360, shea butter, silica, sodium borate, sodium hydroxide, sodium lactate, sodium saccharin, sorbic acid, squalane, stearyl alcohol, sucralose, tetrahexyldecyl ascorbate, theobroma cacao (cocoa) seed butter, titanium dioxide, tocopheryl acetate, tocopheryl acetate (Vitamin E), tocopheryl linoleate, tridecyl neopentanoate, triisononanoin, zea mays (corn) oil, blue 1 lake, D&C red no. 6, barium lake, yellow 10, PPG-5-ceteth-20, 30 lake and so forth.

The lip care formulation can be printed onto the substrate or device in any manner suitable for applying the particular formulation. In addition the lip care formulation may be applied to the entire exterior of the device or wipe, a portion of the device or wipe or in a pattern, for example stripes or dots. The lip care formulation can be applied to a substrate, for example the exterior surface of first section 20 or a portion thereof, at any add-on level which provides the desired transfer benefit. For example, the total add-on level of the lotion formulation may be from about 0.05 to about 100 mg/cm². The add-on amount will depend upon the desired effect of the lip care formulation on the product attributes and the specific lip care formulation. The lip care formulation may be applied to a nonwoven substrate in any of many well known manners. Suggested methods to uniformly apply the lip care formulation to the surface of a nonwoven fabric include spraying and slot coating. These application methods allow control of the formulation distribution and transfer rate. Other application methods, such as rotogravure or flexographic printing, can be used. For example, a lip care formulation may be applied to a nonwoven fabric by (a) heating the lip care formulation to a temperature above the melting point of the formulation, causing the formulation to melt, (b) applying the melted formulation to the fabric surface or a portion of the surface, for example exterior portion of the wipe 22; and (c) resolidifying the deposits of the melted formulation. Desirably, resolidification of the deposits occurs almost instantaneously, without the need for external cooling means such as chill rolls. This can occur if the formulation is heated to a temperature only slightly above or at the melting point of the formulation. However, external means such as chill rolls, either before or after the application of melt, can be used if desired to accelerate resolidification. Increased viscosity of the formulation at the process temperature and the instantaneous resolidification tends to impede penetration of the formulation into the nonwoven fabric and retain it on the exterior surface of a dental wipe 10 or a device 2, which is advantageous. For example, the temperature of the melted formulation can advantageously be less than about 10° C., more desirably less than about 5° C., and still more desirably less than about 2° C. above the melting point of the formulation prior to applying it to the surface of the nonwoven fabric for reduced migration. As the temperature of the melted formulation approaches the melting point of the formulation, the viscosity of the melted formulation generally increases, which further enhances the tendency of the melted formulation to be retained on the surface.

A dental wipe of the present invention may also include one or more dental agents. The portion of the wipe that includes the dental agent should be the same portion of the dental wipe that is intended to be used to clean teeth and or gum surfaces. The portion of the wipe that is intended for dental cleaning may be the same surface as the portion that is intended to include the lip care formulation or may overlap with the portion of the wipe that will be used to apply the lip care formulation. However, it is suggested the that portion of the dental wipe that will include the lip care formulation differs from the portion of the dental wipe that will be used to clean teeth so that lip care formulation will be available on the surface of the dental wipe after the dental wipe has been used to clean teeth. Suggested dental agents include, but are not limited to, desensitizing agents, fluorides, whitening agents and so forth. Suggested desensitizing agents include, but are not limited to potassium nitrate. Suggested fluorides include, but are not limited to, sodium fluoride, sodium monofluorophosphate, stannous fluoride and WN fluoride ion. Suggested whitening agents include, but are not limited to, carbamide peroxide, hydrogen peroxide, calcium peroxide and various other peroxides. The dental wipe may further include one or more breath freshening agents. Breath freshening agents are known and include, but are not limited to, peppermint oil, spearmint oil, cinnamon bark oil and so forth. Flavoring agents are also known and include, but are not limited to, various flavor, flavors, fragrances, fruit extracts, and so forth. Suggested flavoring agents include various essential oils, such as peppermint oil, spearmint oil, cinnamon oils, orange oil and so forth.

In general, devices of the present invention, such as depicted in FIGS. 2-5, can be formed from variety of materials. For instance, as stated above, in one embodiment, the dental wipe can be formed as a unitary structure from a base web. In another embodiment, the dental wipe can be formed from two sections made from the same or different base webs. It should be understood, however, that, as used herein, a base web of the present invention is meant to include one or more layers of fibrous materials. Generally, a base web of the present invention can contain any material used in the art for making wipes. For most applications, dental wipes made in accordance with the present invention are constructed from nonwoven webs containing an elastic component referred to herein as an “elastic nonwoven”. An elastic nonwoven is a nonwoven material having nonelastic and elastic components or having purely elastic components.

The elastic component can form a separate section of the dental wipe. For example, the dental wipe can be made from two or more sections of material that includes a first section made from a non-elastic material and a second section made from an elastic material. The non-elastic material can be used to clean the teeth, gums and tongue of the user, while the elastic material can be used to ensure that the dental wipe fits snugly over the finger of the user. In one embodiment, the non-elastic material can be texturized for cleaning the teeth, gums and tongue, while the elastic material can have a smooth surface for use in polishing the teeth, gums and tongue of the user. Alternatively, the dental wipe can be made from a single piece of an elastic nonwoven. The elastic component contained in the elastic nonwoven can be a film, strands, a nonwoven web or elastic filaments incorporated into a laminate structure that is well suited to cleaning or scrubbing one's teeth.

Non-elastic materials used in the present invention typically include nonwoven webs or films. The nonwoven webs, for instance, can be meltblown webs, spunbond webs, carded webs, and the like. The webs can be made from various fibers, such as synthetic or natural fibers. For instance, in one embodiment, synthetic fibers, such as fibers made from thermoplastic polymers, can be used to construct the dental wipe of the present invention. For example, suitable fibers could include melt-spun filaments, staple fibers, melt-spun multi-component filaments, and the like. The synthetic fibers or filaments used in making the nonwoven material of the base web may have any suitable morphology and may include hollow or solid, straight or crimped, single component, conjugate or biconstituent fibers or filaments, and blends or mixtures of such fibers and/or filaments, as are well known in the art.

The synthetic fibers used in the present invention may be formed from a variety of thermoplastic polymers where the term “thermoplastic polymer” refers to a long chain polymer that repeatedly softens when exposed to heat and substantially returns to its original state when cooled to ambient temperature. As used herein, the term “polymer” generally includes, but is not limited to, homopolymers, copolymers, such as for example, block, graft, random, and alternating copolymers, terpolymers, etc., and blends and modifications thereof. As used herein, the term “blend” means a mixture of two or more polymers. Furthermore, unless otherwise specifically limited, the term “polymer” shall include all possible geometrical configurations of the molecule. These configurations include, but are not limited to, isotactic, syndiotactic, and random symmetries.

Exemplary thermoplastics include, without limitation, poly(vinyl) chlorides, polyesters, polyamides, polyfluorocarbons, polyolefins, polyurethanes, polystyrenes, poly(vinyl) alcohols, caprolactams, and copolymers of the foregoing, and elastomeric polymers such as elastic polyolefins, copolyether esters, polyamide polyether block copolymers, ethylene vinyl acetates (EVA), block copolymers having the general formula A-B-A′ or A-B like copoly(styrene/ethylene-butylene), styrene-poly(ethylene-propylene)-styrene, styrene-poly(ethylene-butylene)-styrene, (polystyrene/poly(ethylene-butylene)/polystyrene, poly(styrene/ethylene-butylene/styrene), A-B-A-B tetrablock copolymers and the like.

Many polyolefins are available for fiber production, for example polyethylenes such as Dow Chemical's PE XU 61800.41 linear low density polyethylene (“LLDPE”) and 25355 and 12350 high density polyethylene (“HDPE”) are such suitable polymers. Fiber-forming polypropylenes include Exxon Chemical Company's Escorene7 PD 3445 polypropylene and Montell Chemical Co.'s PF-304 and PF-015. Many other polyolefins are commercially available and include polybutylenes and others.

Examples of polyamides and their methods of synthesis may be found in “Polymer Resins” by Don E. Floyd (Library of Congress Catalog No. 66-20811, Reinhold Publishing, New York, 1966). Particularly commercially useful polyamides are nylon-6, nylon 6,6, nylon-11 and nylon-12. These polyamides are available from a number of sources such as Emser Industries of Sumter, S.C. (Grilon7 & Grilamid7 nylons), Atochem Inc. Polymers Division of Glen Rock, N.J. (Rilsan7 nylons), Nyltech of Manchester, N.H. (grade 2169, Nylon 6), and Custom Resins of Henderson, Ky. (Nylene 401-D), among others.

As stated above, synthetic fibers added to the base web can also include staple fibers which can be added to increase the strength, bulk, softness and smoothness of the base sheet. Staple fibers can include, for instance, various polyolefin fibers, polyester fibers, nylon fibers, polyvinyl acetate fibers, cotton fibers, rayon fibers, non-woody plant fibers, and mixtures thereof. In general, staple fibers are typically longer than pulp fibers. Staple fibers can increase the strength and softness of the final product. The fibers used in the base web of the present invention can also be curled or crimped. The fibers can be curled or crimped, for instance, by adding a chemical agent to the fibers or subjecting the fibers to a mechanical process. Curled or crimped fibers may create more entanglement and void volume within the web and further increase the amount of fibers oriented in the z-direction as well as increase web strength properties. As used herein, the z-direction refers to the direction perpendicular to the length and width of the base web.

The synthetic fibers added to the base web can also include bicomponent fibers. Bicomponent fibers are fibers that can contain two materials such as but not limited to in a side by side arrangement, in a matrix-fibril arrangement wherein a core polymer has a complex cross-sectional shape, or in a core and sheath arrangement. In a core and sheath fiber, generally the sheath polymer has a lower melting temperature than the core polymer to facilitate thermal bonding of the fibers. For instance, the core polymer, in one embodiment, can be a nylon or a polyester polymer, while the sheath polymer can be a polyolefin such as polyethylene or polypropylene. Such commercially available bicomponent fibers include “CELBOND” fibers marketed by the Hoechst Celanese Company.

Besides or in addition to synthetic fibers, pulp fibers can also be used to construct the dental wipe of the present invention. The pulp fibers used in forming the base web may be softwood fibers having an average fiber length of greater than 1 mm, and particularly from about 2 to 5 mm based on a length-weighted average. Such fibers can include Northern softwood kraft fibers, redwood fibers and pine fibers. Secondary fibers obtained from recycled materials may also be used. In addition, hardwood pulp fibers, such as eucalyptus fibers, can also be utilized in the present invention.

Besides the above-mentioned fibers, thermomechanical pulp fibers can also be added to the base web. Thermomechanical pulp, as is known to one skilled in the art, refers to pulp that is typically cooked during the pulping process to a lesser extent than conventional pulps. Thermomechanical pulp tends to contain stiff fibers and has higher levels of lignin. Thermomechanical pulp can be added to the base web of the present invention in order to create an open pore structure, thus increasing bulk and absorbency and improving resistance to wet collapse. When present, thermomechanical pulp can be added to a layer of the base web in an amount from about 10 percent to about 30 percent by weight of the fibers contained in the layer. When using thermomechanical pulp, a wetting agent may be added during formation of the web. The wetting agent can be added in an amount less than about 1 percent by weight of the fibers. In general, any suitable wetting agent can be used in the present invention. For example, in one embodiment, the wetting agent can be a sulphonated glycol.

When pulp fibers are used to form the base web, the web can be treated with a chemical debonding agent to reduce inner fiber-to-fiber strength. Suitable debonding agents that may be used in the present invention when the base web contains pulp fibers include cationic debonding agents such as fatty dialkyl quaternary amine salts, mono fatty alkyl tertiary amine salts, primary amine salts, imidazoline quaternary salts, and unsaturated fatty alkyl amine salts. Other suitable debonding agents are disclosed in U.S. Pat. No. 5,529,665 to Kaun, which is incorporated herein by reference. In one embodiment, the debonding agent can be an organic quaternary ammonium chloride. In this embodiment, the debonding agent can be added to the fiber furnish in an amount from about 0.1 percent to about 1 percent by weight, based on the total weight of fibers present within the furnish.

Moreover, in some embodiments of the present invention, the base web of the present invention can also be hydraulically entangled (or hydroentangled) to provide further strength. Hydroentangled webs, which are also known as spunlace webs, refer to webs that have been subjected to columnar jets of a fluid that cause the fibers in the web to entangle. Hydroentangling a web typically increases the strength of the web. Thus, according to the present invention, in order to increase the strength of a web, the base web of the present invention can be hydroentangled. For example, in one embodiment, the base web can comprise HYDROKNIT7, a nonwoven composite fabric that contains 70 percent by weight pulp fibers that are hydraulically entangled into a continuous filament material. HYDROKNIT7 material is commercially available from Kimberly-Clark Corporation of Neenah, Wis. Hydraulic entangling may be accomplished utilizing conventional hydraulic entangling equipment such as may be found in, for example, in U.S. Pat. No. 3,485,706 to Evans or U.S. Pat. No. 5,389,202 to Everhart et al., the disclosures of which are hereby incorporated by reference.

As mentioned above, for most applications, nonwoven webs used to construct the dental wipe will contain synthetic fibers. For nonwoven webs containing substantial amounts of synthetic fibers, the webs may be bonded or otherwise consolidated in order to improve the strength of the web. Various methods may be utilized in bonding webs of the present invention. Such methods include through-air bonding and thermal point bonding as described in U.S. Pat. No. 3,855,046 to Hansen et al, which is incorporated herein by reference. In addition, other conventional means of bonding, such as oven bonding, ultrasonic bonding, hydroentangling, or combinations of such techniques, may be utilized in certain instances.

In one embodiment, thermal point bonding is used which bonds the fibers together according to a pattern. In general, the bonding areas for thermal point bonding, whether pattern unbonded or pattern bonded fabrics, can be in the range of 50 percent total bond area or less. More specifically, the bond areas of the present inventive webs can be in the range of about 40 percent total bond area or less. Even more specifically, the bond areas can be in the range of about 30 percent total bond area or less and may be in the range of about 15 percent total bond area or less. Typically, a bond area of at least about 10 percent can be acceptable for creating the base webs of the present invention, although other total bond areas will fall within the scope of the invention, depending on the particular characteristics desired in the final product. Stated generally, the lower limit on the percent bond area suitable for forming the nonwoven material of the present invention is the point at which fiber pull-out reduces the surface integrity and durability of the material. The percent bond areas will be affected by a number of factors, including the type(s) of polymeric materials used in forming the fibers or filaments of the nonwoven web, whether the nonwoven web is a single- or multi-layer fibrous structure, and the like. Bond areas ranging from about 15 percent to about 50 percent, and more particularly from about 15 percent to about 40 percent, have been found suitable.

Base webs constructed for use in the dental wipe of the present invention desirably include a texturized surface where the dental wipe is to contact a user's teeth and gums. The texturized surface can facilitate removal of residue and film from the teeth and gums. The texturized surface can be positioned on the dental wipe only where the dental wipe is to contact the teeth and gums or can completely cover the exterior surface of the dental wipe. In this regard, referring to FIG. 3, one embodiment of the present invention includes a second section 30 that is made from a base web comprising a nonwoven texturized material. In particular, when the dental wipe 10 is placed onto a finger, as shown in FIG. 2, the second section 30 can be used in order to clean and/or massage the teeth or gums of the desired subject.

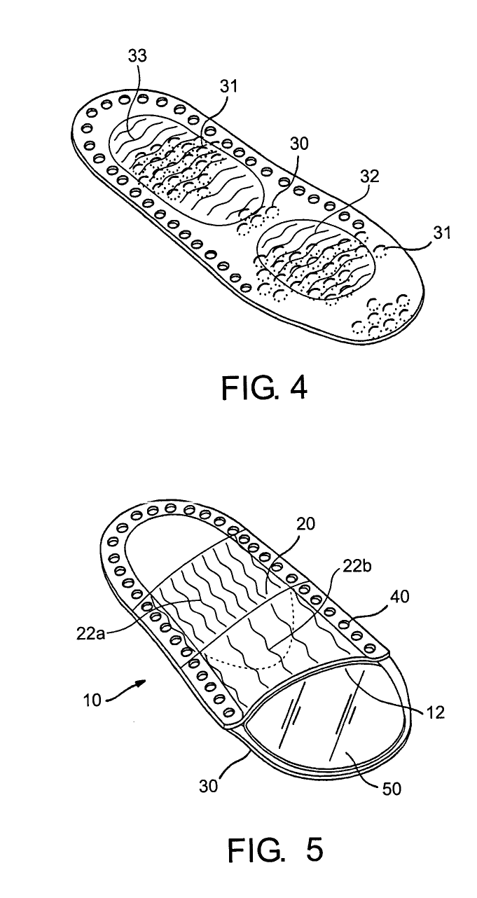

The manner in which a texturized surface is formed on a nonwoven web for use in the present invention can vary depending upon the particular application and the desired result. In the embodiment shown in FIG. 4, the second section 30 is made from a nonwoven web that has been thermally point unbonded to form a plurality of tufts 31. As used herein, a substrate that has been “thermally point unbonded” refers to a substrate that includes raised unbonded areas or lightly bonded areas that are surrounded by bonded regions. For example, as shown in FIG. 4, the tufts 31 are the unbonded or lightly bonded areas that form raised projections off the surface of the nonwoven web to provide the necessary texture. Processes for producing thermally point unbonded substrates are known. One process for producing is described in U.S. Patent Application Publication no. 2002/0170133 A1 to McDevitt et al. The total bond area surrounding the tufts 31 can also vary depending upon the particular application. For most embodiments, the bond area surrounding the tufts can be from about 15 percent to about 40 percent of the surface area of the material, and particularly from about 20 percent to about 40 percent of the surface area of the material.

Besides point unbonded materials, there are many other methods for creating texturized surfaces on base webs and many other texturized materials can be utilized. Examples of known nonwoven, texturized materials, include rush transfer materials, flocked materials, wireform nonwovens, and the like. Moreover, through-air bonded fibers, such as through-air bonded bicomponent spunbond, can be incorporated into the base web to provide texture to the wipe.

Texturized webs having projections from about 0.1 mm to about 25 mm, such as pinform meltblown or wireform meltblown, can also be utilized in the base web of the present invention. Still another example of suitable materials for a texturized base web includes texturized coform materials. In general, “coform” means a process in which at least one meltblown in die is arranged near a chute through which other materials are added to the web while it forms. Such other materials can include, for example, pulp, superabsorbent particles, or cellulose or staple fibers. Coform processes are described in U.S. Pat. No. 4,818,464 to Lau and U.S. Pat. No. 4,100,324 to Anderson et al., which are incorporated by reference. Webs produced by the coform process are generally referred to as coform materials.

In one embodiment, the texturized material can be a loop material. As used herein, a loop material refers to a material that has a surface that is at least partially covered by looped bristles. It is believed that looped bristles provide various advantages in relation to conventional bristles. For example, the inherent stiffness in a looped structure allows the use of finer yarns and a corresponding increase in surface area for a given stiffness. The lack of a sharp end on a looped bristle may reduce abrasion, which refers to the damage that can occur to soft tissue in the mouth.

The looped bristles that can be used in the present invention can vary depending upon the particular application. For instance, the stiffness of the looped bristles can be varied by varying different factors, including the height of the loop, the inherent properties of the looped material, the fiber diameter, the fiber type, and any post-formation treatments (e.g. chemical coatings) that may be performed on the looped material.

In general, the height of the looped bristles should be short enough so that the loops are unlikely to get snagged on teeth or dental work, but still sufficiently long to be effective in cleaning the interproximal areas of the teeth. For most applications, the loops should be less than about 20 mm, particularly from about 1 mm to about 5 mm, and more particularly from about 1.5 mm to about 3.5 mm. The height of the looped bristles on a loop material can be homogenous or heterogeneous. The looped bristles can be contained on the looped material according to a particular pattern or can be randomly arranged on the loop material. For example, in one embodiment, the looped bristles can be arranged in rows and columns on the loop material. The looped bristles can be arranged vertically or at any suitable angle to the surface of the material. Further, the looped bristles can be sparsely spaced apart or can be densely packed together.

The loop material can be made in a number of different ways. For example, the loop can be a woven fabric or a knitted fabric. In one embodiment, the loop material is made by needle punching loops into a substrate. In other embodiments, the loop material can be formed through a hydroentangling process or can be molded, such as through an injection molding process. Of course, any other suitable technique known in the art for producing looped bristles can also be used. The looped bristles can be made from various natural or synthetic materials. For instance, the bristles can be made from polyester, nylon, polypropylene, polyethylene, polylactic acid, or various other polymers. The looped bristles can also be made from natural fibers, including cotton or wool. The looped bristles can be made from monofilament yarns, multi-filament yarns, or spun yarns. Further, the yarns can be shaped filaments, such as a multi-lobal shaped filament. As used herein “shaped” filaments or fibers refer to filaments or fibers not having a circular cross sectional shape. For example, a pentalobal filament can be used. In accordance with the present invention, the looped bristles can be flavored or unflavored. Further, the looped bristles can be treated, such as with a fluoride compound or other additive described herein, or untreated.

Further, the looped bristles can be made from the same material as the base material on which the bristles are contained or can be made from a different material. For example, as described above, the bristles can be needle punched into a woven or non-woven backing layer. The loop material can also be made from a single layer of material or can be a laminate. For example, a base layer containing the looped bristles can be laminated to various other layers. For example, the base layer can be laminated to a woven layer, a knitted layer, a non-woven layer, an expandable layer such as spandex, a stretch bonded layer, or a neck bonded layer, or can be attached to various non-woven webs including spunbonded webs or spunbond-meltblown-spunbond laminate.

As described above, besides containing various non-elastic materials and, if desired, a texturized surface, the dental wipe of the present invention can also contain an elastomeric component. By containing such an elastomeric component, the dental wipe of the present invention can better fit around a human finger. In this regard, referring to FIG. 3, one embodiment of the present invention is depicted that includes a dental wipe made from a base web having at least one elastomeric component. In particular, the dental wipe 10 can be formed into a unitary structure from a base web that includes an elastomeric material. In FIG. 3, section 20 of the dental wipe can include an elastomeric component.

When present in the dental wipe, the elastomeric component can take on various forms. For example, the elastomeric component can be elastic strands or sections uniformly or randomly distributed throughout the base web. Alternatively, the elastomeric component can be an elastic film or an elastic nonwoven web. The elastomeric component can also be a single layer or a multi-layered material.

In general, any material known in the art to possess elastomeric characteristics can be used in the present invention as an elastomeric component. For example, suitable elastomeric resins include block copolymers having the general formula A-B-A′ or A-B, where A and A′ are each a thermoplastic polymer endblock which contains a styrenic moiety such as a poly(vinyl arene) and where B is an elastomeric polymer midblock such as a conjugated diene or a lower alkene polymer. Block copolymers form the A and A′ blocks, and the present block copolymers are intended to embrace linear, branched and radial block copolymers. In this regard, the radial block copolymers may be designated (A-B)ₘ—X, wherein X is a polyfunctional atom or molecule and in which each (A-B)ₘ— radiates from X in a way that A is an endblock. In the radial block copolymer, X may be an organic or inorganic polyfunctional atom or molecule and m is an integer having the same value as the functional group originally present in X. It is usually at least 3, and is frequently 4 or 5, but not limited thereto. Thus, in the present invention, the expression “block copolymer,” and particularly “A-B-A” and “A-B” block copolymer, is intended to embrace all block copolymers having such rubbery blocks and thermoplastic blocks as discussed above, which can be extruded (e.g., by meltblowing), and without limitation as to the number of blocks. The elastomeric nonwoven web may be formed from, for example, elastomeric (polystyrene/poly(ethylene-butylene)/polystyrene) block copolymers. Commercial examples of such elastomeric copolymers are, for example, those known as KRATON materials which are available from Shell Chemical Company of Houston, Tex. KRATON block copolymers are available in several different formulations, a number of which are identified in U.S. Pat. Nos. 4,663,220, 4,323,534, 4,834,738, 5,093,422 and 5,304,599, which are all hereby incorporated by reference.

Polymers composed of an elastomeric A-B-A-B tetrablock copolymer may also be used in the practice of this invention. Such polymers are discussed in U.S. Pat. No. 5,332,613 to Taylor et al. In such polymers, A is a thermoplastic polymer block and B is an isoprene monomer unit hydrogenated to substantially a poly(ethylene-propylene) monomer unit. An example of such a tetrablock copolymer is a styrene-poly(ethylene-propylene)-styrene-poly(ethylene-propylene) or SEPSEP elastomeric block copolymer available from the Shell Chemical Company of Houston, Tex. under the trade designation KRATON G-1657.

When incorporating an elastomeric component, such as described above, into a base web of the present invention, it is often desired that the elastomeric material form an elastic laminate with one or more other layers, such as foams, films, apertured films, and/or nonwoven webs. The elastic laminate generally contains layers that can be bonded together so that at least one of the layers has the characteristics of an elastic polymer. Examples of elastic laminates include, but are not limited to, stretch-bonded laminates and neck-bonded laminates.

The elastic member used in neck bonded materials, stretch-bonded materials, stretch-bonded laminates, neck-bonded laminates and in other similar laminates can be made from materials, such as described above, that are formed into films, such as a microporous film, fibrous webs, such as a web made from meltblown fibers, or foams. A film, for example, can be formed by extruding an elastomeric polymer containing a filler, such as calcium carbonate, and subsequently stretching it to render it microporous.

Fibrous elastic webs can also be formed from an extruded polymer. For instance, as stated above, in one embodiment the fibrous web can contain meltblown fibers. The fibers can be continuous or discontinuous. Meltblown fabrics have been conventionally made by extruding a thermoplastic polymeric material through a die to form fibers. As the molten polymer fibers exit the die, a high pressure fluid, such as heated air or steam, attenuates the molten polymer filaments to form fine fibers. Surrounding cool air is induced into the hot air stream to cool and solidify the fibers. The fibers are then randomly deposited onto a foraminous surface to form a web. The web has integrity but may be additionally bonded if desired.

Besides meltblown webs, however, it should be understood that other fibrous webs can be used in accordance with the present invention. For instance, in an alternative embodiment, elastic spunbond webs can also be formed from spunbond fibers. Spunbond webs are typically produced by heating a thermoplastic polymeric resin to at least its softening temperature, then extruding it through a spinnerette to form continuous fibers, which can be subsequently fed through a fiber draw unit. From the fiber draw unit, the fibers are spread onto a foraminous surface where they are formed into a web and then bonded such as by chemical, thermal or ultrasonic means.

In one embodiment, the elastic member can be a necked stretch bonded laminate. As used herein, a necked stretch bonded laminate is defined as a laminate made from the combination of a neck bonded laminate and a stretch bonded laminate. Examples of necked stretch bonded laminates are disclosed in U.S. Pat. Nos. 5,114,781 and 5,116,662 which are both incorporated herein by reference. Of particular advantage, a necked stretch bonded laminate is stretchable in the machine direction and in the cross machine direction. Further, a necked stretch bonded laminate can be made with a nonwoven facing that is texturized. In particular, the necked stretch bonded laminate can be made so as to include a nonwoven facing that gathers and becomes bunched so as to form a texturized surface. In this manner, the necked stretch bonded laminate can be used to form the entire dental wipe having stretch characteristics in two directions and having a texturized surface for cleaning the teeth and gums of a user.

Besides including a non-elastic component or an elastic component, the dental wipe of the present invention can further include a moisture barrier that is incorporated into or laminated to the base web of the present invention. Such a barrier can prevent, or at least minimize, leakage from outside the wipe by establishing a barrier to the passage of liquid from the wipe to the finger placed therein. For example, as shown in FIG. 5, a layer of material or film can be provided to form the moisture barrier 50, which can act as a barrier between the outer layer of a wipe 10 and a finger. Moreover, in this embodiment, the moisture barrier 50 can act as an inner lining for the second section 30 only, while the first section 20 possesses no such inner lining. However, it should also be understood that the moisture barrier 50 may be a liner for both the first section 20 and the second section 30. It should be understood that the moisture barrier 50 can be applied to the wipe 10 as a layer of the base web, or as an outer lining for the base web. Moreover, it should also be understood that the moisture barrier can be inherent within the base web structure such that it would not constitute a separate lining thereof.

In one particular application, the moisture barrier layer can be used to secure the bristles to a base web. For example, in one embodiment, bristles or looped bristles can be needle punched into a base web. According to this process, holes may form in the base web that would allow liquids to pass from the surface of the dental wipe to the interior of the dental wipe. In this application, the moisture barrier layer can be applied as an inner lining to the base web for not only making the base web liquid impervious, but for also securing the bristles to the surface of the base web.

In one embodiment of the present invention, the moisture barrier 50 can be made from liquid-impermeable plastic films, such as polyethylene and polypropylene films. Generally, such plastic films are impermeable to gases and water vapor, as well as liquids. While completely liquid-impermeable films can prevent the migration of liquid from outside the wipe to the finger, the use of such liquid- and vapor-impermeable barriers can sometimes result in a relatively uncomfortable level of humidity being maintained in a wipe 10.

As such, in some embodiments, breathable, liquid-impermeable barriers are desired. For instance some suitable breathable, liquid-impermeable barriers can include barriers such as disclosed in U.S. Pat. No. 4,828,556 to Braun et al., which is incorporated herein in its entirety by reference. The breathable barrier of Braun et al. is a multilayered, clothlike barrier comprised of at least three layers. The first layer is a porous nonwoven web; the second layer, which is joined to one side of the first layer, comprises a continuous film of PVOH; and the third layer, which is joined to either the second layer or the other side of the first layer not joined with the second layer, comprises another porous nonwoven web. The second layer continuous film of PVOH is not microporous, meaning that it is substantially free of voids which connect the upper and lower surfaces of the film.

In other cases, various films can be constructed with micropores therein to provide breathability. The micropores form what is often referred to as tortuous pathways through the film. Liquid contacting one side of the film does not have a direct passage through the film. Instead, a network of microporous channels in the film prevents water from passing, but allows water vapor to pass.

In some instances, the breathable, liquid-impermeable barriers are made from polymer films that contain any suitable substance, such as calcium carbonate. The films are made breathable by stretching the filled films to create the microporous passageways as the polymer breaks away from the calcium carbonate during stretching. In some embodiments, the breathable film layers can be used in thicknesses of from about 0.01 mils to about 5 mils, and in other embodiments, from about 0.01 mils to about 1.0 mils.

An example of a breathable, yet fluid penetration-resistant material is described in U.S. Pat. No. 5,591,510 to Junker et al., which is incorporated herein by reference. The fabric material described in Junker et al. contains a breathable outer layer of paper stock and a layer of breathable, fluid-resistant nonwoven material. The fabric also includes a thermoplastic film having a plurality of perforations which allow the film to be breathable while resisting direct flow of liquid therethrough.

In addition to the films mentioned above, various other breathable films can be utilized in the present invention. One type of film that may be used is a nonporous, continuous film, which, because of its molecular structure, is capable of forming a vapor-permeable barrier. Among the various polymeric films which fall into this type include films made from a sufficient amount of poly(vinyl alcohol), polyvinyl acetate, ethylene vinyl alcohol, polyurethane, ethylene methyl acrylate, and ethylene methyl acrylic acid to make them breathable. Although the inventors do not intend to be held to a particular mechanism of operation, it is believed that films made from such polymers solubilize water molecules and allow transportation of those molecules from one surface of the film to the other. Accordingly, such films may be sufficiently continuous, i.e., nonporous, to make them liquid-impermeable but still allow for vapor permeability.

Still, other breathable, liquid-impermeable barriers that can be used in the present invention are disclosed in U.S. patent application Ser. No. 08/928,787 entitled “Breathable, Liquid-Impermeable, Apertured Film/Nonwoven Laminate and Process for Making the Same”, which is incorporated herein in its entirety by reference. For example, breathable films and/or apertured films can be utilized in the present invention. Such films can be included within a laminate structure. In one embodiment, a breathable, liquid-impermeable, apertured film/nonwoven laminate material can be formed from a nonwoven layer, an apertured film layer, and a breathable film layer. The layers may be arranged so that the apertured film layer or the breathable film layer is attached to the nonwoven layer.

For instance, in one embodiment, an apertured film can be used in the present invention that is made from any thermoplastic film, including polyethylene, polypropylene, copolymers of polypropylene or polyethylene, or calcium carbonate-filled films. The particular aperturing techniques utilized to obtain the apertured film layer may be varied. The film may be formed as an apertured film or may be formed as a continuous, non-apertured film and then subjected to a mechanical aperturing process.

Moisture barrier layers, as described above, can be used alone or incorporated into a laminate when used to construct the dental wipe of the present invention. When incorporated into a laminate, the laminate can include various nonwoven webs in combination with the moisture barrier layer. For instance, moisture barrier laminates can be formed from many processes such as for example, meltblowing processes, spunbonding processes, coforming processes, spunbonding/meltblowing/spunbonding processes (SMS), spunbonding/meltblowing processes (SM), and bonded carded web processes. For instance, in one embodiment, the nonwoven layer of a laminate moisture barrier of the present invention is a spunbond/meltblown/spunbond (SMS) and/or spunbond/meltblown (SM) material. An SMS material is described in U.S. Pat. No. 4,041,203 to Brock et al. which is incorporated herein in its entirety by reference. Other SMS products and processes are described for example in U.S. Pat. No. 5,464,688 to Timmons et al., U.S. Pat. No. 5,169,706 to Collier et al. and U.S. Pat. No. 4,766,029 to Brock et al., all of which are also incorporated herein in their entireties by reference. Generally, an SMS material will contain a meltblown web sandwiched between two exterior spunbond webs. Such SMS laminates are available from Kimberly-Clark Corporation under marks such as Spunguard7 and Evolution7. The spunbonded layers on the SMS laminates provide durability and the internal meltblown barrier layer provides porosity and additional clothlike feel. Similar to an SMS laminate, an SM laminate is essentially a spunbond layer laminated to a meltblown layer.

In forming a dental wipe of the present invention with a moisture barrier, the barrier can be bonded together with the other layers of the wipe in a number of various ways. Thermal bonding, adhesive bonding, ultrasonic bonding, extrusion coating, and the like, are merely examples of various bonding techniques that may be utilized in the present process to attach the moisture barrier to the fibrous layers of the dental wipe.

In some embodiments, any of the above layers and/or materials can also be dyed or colored so as to form a base web or moisture barrier having a particular color. For example, in one embodiment, the moisture barrier can be provided with a colored background. For instance, white tufts, colored tufts, and/or a white titanium oxide background could be utilized.

In order to provide better dental care to the teeth being cleaned, a variety of chemical additives can be applied to the dental wipe of the present invention. For example, in one embodiment, cationic polymers can be coated onto the dental wipe. Cationic polymers can help clean teeth and/or gums because they typically have a strong attraction for negatively charged bacteria and deleterious acidic by-products that accumulate in plaque. One example of a cationic polymer that is suitable for use in the present invention is chitosan (poly-N-acetylglucosamine, a derivative of chitin) or chitosan salts. Chitosan and its salts are natural biopolymers that can have both hemostatic and bacteriostatic properties. As a result, chitosan can help reduce bleeding, reduce plaque, and reduce gingivitis.

In addition to chitosan and chitosan salts, any other cationic polymer known in the art can generally be applied to a dental wipe of the present invention. For example, in one embodiment, cationic starches are used in the present invention. One such suitable cationic starch is, for example, COBOND, which can be obtained from National Starch. In another embodiment, cationic materials that are oligomeric compounds can be used. In some embodiments, combinations of cationic materials can be utilized.

In addition to the chemical additives mentioned above, a variety of other additives can be applied to a dental wipe of the present invention. For instance, other well known dental agents can be utilized. Examples of such dental agents include, but are not limited to alginates, soluble calcium salts, phosphates, fluorides, such as sodium fluoride (NaF) or stannous fluoride (SnF2), and the like. Moreover, mint oils and mint oil mixtures can be applied to the dental wipe of the present invention. For instance, in one embodiment, peppermint oil can be applied to the dental wipe. Moreover, in another embodiment, a mint oil/ethanol mixture can be applied. Components of mint oil (e.g., menthol, carvone) can also be used. Additionally, various whitening agents can be applied to the dental wipe. Examples of whitening agents include silica, peroxides and in situ sources of peroxide, such as carbamide peroxide.

Furthermore, in some embodiments, the dental wipe can also comprise an anti-ulcer component. In particular, one embodiment of the present invention can comprise a component designed to act as an anti-H. pylori agent. In general, any additive known in the art to be an anti-ulcer or anti-H. pylori agent can be used in the present invention. In one embodiment, for example, bismuth salts can be utilized. One particularly effective bismuth salt, bismuth subcitrate, is described in more detail in U.S. Pat. No. 5,834,002 to Athanikar, which is incorporated herein in its entirety by reference thereto. Another example of a suitable bismuth salt is PEPTO-BISMOL sold by The Procter & Gamble Company, containing bismuth subsalicylate. In addition to bismuth salts, other examples of suitable anti-ulcer additives include, but are not limited to, tetracycline, erythromycin, clarithromycin, omeprazole, metronidazole, or other antibiotics. Furthermore, any additive useful for treating peptic ulcers, such as H2-blockers, sucralfate, and the like, can be used as well.

Besides the above additives, other additives can also be applied to the dental wipe. Such materials can include, but are not limited to, flavoring agents, preservatives, anti-microbial agents, polishing agents, hemostatic agents, anti-plaque agents, anti-caries agents, antibiotics, antioxidants, desensitizers, lubricants, remineralization agents, tartar control agents, surfactants, etc. Examples of suitable flavoring agents include various sugars, breath freshening agents, menthol, carvone, anise oil, anethole, methyl salicylate, thymol and artificial sweeteners as well as natural flavorants, cinnamon, vanilla and citrus. Moreover, in one embodiment, xylitol, which provides a cooling effect upon dissolution in the mouth and is anti-cariogenic, can be used as the flavoring agent. As stated, preservatives, such as methyl benzoate or methyl paraben, can also be applied to a dental wipe of the present invention.

Suggested polishing agents include but are not limited to, silica and sodium bicarbonate. Suggested anti-microbial agents or anti-bacterial agents include, but are not limited to, triclosan, thymol, eucalyptol, menthol, methyl salicylate, chlorhexidine, hexetidine, hydrogen peroxide and carbamide peroxide. Suggested anti-caries agents include, but are not limited to, triclosan, xylitol, and fluoride. Suggested anti-plaque agents include, but are not limited to, triclosan, menthol, thymol, eucalyptol, methyl salicylate and cetylpyridinium chloride.

In addition, a variety of other additives and combinations thereof can be applied to a dental wipe of the present invention. For instance, examples of various materials that can be utilized as additives in the present invention are described in U.S. Pat. No. 3,902,509 to Tundermann et al. and U.S. Pat. No. 5,445,825 to Copelan et al., which are incorporated herein by reference. Although various specific additives have been specifically mentioned above, it should be understood that any additive can generally be applied to the dental wipe of the present invention. The additives can be applied to the dental wipe as is or they can be encapsulated in order to preserve the additives and/or to provide the additive with time release properties.

In general, the chemical additives described above can be applied to a dental wipe of the present invention according to a number of ways known in the art. For example, the additives can be applied to the wipe using a saturant system, such as disclosed in U.S. Pat. No. 5,486,381 to Cleveland et al., which is incorporated herein by reference. Moreover, the additives can also be applied by various other methods, such as print, blade, roll, spray, spray-drying, foam, clean treating applications, etc., which are well known in the art. The additives can further be applied as a mixture of molten solids or co-extruded onto the wipe. Additionally, in another embodiment, the chemical additives can be impregnated into the material during manufacturing as is well known in the art. It should be understood that when coated onto a wipe as described above, the additives can be applied to the base web before or after the base web is stamped or bonded to form the dental wipe of the present invention. Furthermore, it should also be understood that, if desired, various additives, solutions, and chemicals can be applied by the consumer to the wipe just before use.

In another embodiment, the additive is encapsulated and then applied to the dental wipe. Encapsulation is a process by which a material or mixture of materials is coated with or entrapped within another material or mixture of materials. The technique is commonly used in the food and pharmaceutical industries. The material that is coated or entrapped is normally a liquid, although it can also be a solid or gas, and is referred to herein as the core material. The material that forms the coating is referred to as the carrier material. A variety of encapsulation techniques are well-known in the art and can be used in the current invention, including spray drying, spray chilling and cooling, coacervation, fluidized bed coating, liposome entrapment, rotational suspension separation, and extrusion.

Spray drying is commonly used for encapsulating food and flavors. To prepare a material for spray drying, the carrier material is dissolved in an aqueous solution. The core ingredient is added to this solution and mixed thoroughly. A typical load of carrier to core material is 4:1, although much higher or lower loads can be used. The mixture is homogenized, and then fed into a spray dryer where it is atomized and released into a stream of hot air. The water is evaporated, leaving a dried particle comprising the core material trapped within the carrier matrix.

Suitable carrier materials include but are not limited to gums, gum Arabic, modified starches, gelatin, cellulose derivatives, and maltodextrins. Suitable core materials include but are not limited to flavors, natural oils, additives, sweeteners, stabilizers besides the other various additives mentioned above.

Regardless of the mechanism utilized to apply the chemical additives to the wipe, the additives can be applied to the wipe via an aqueous solution, non-aqueous solution, oil, lotion, cream, suspension, gel, etc. When utilized, an aqueous solution can contain any of a variety of liquids, such as various solvents and/or water. Moreover, the solution can often contain more than one additive. In some embodiments, the additives applied by an aqueous solution or otherwise constitute approximately less than 80 percent by weight of the dental wipe. In other embodiments, the additives can be applied in an amount less than about 50 percent of the weight of the wipe.

Moreover, in some embodiments, the additives can also be applied asymmetrically onto the wipe to reduce costs and maximize performance of the wipe. For instance, in one embodiment, a flat sheet of the base web is asymmetrically contacted with a particular coating agent, and thereafter stamped and bonded to form the dental wipe of the present invention, wherein only the surface used to clean teeth is coated with the additives. In another embodiment, the finger wipe is stamped and bonded, and thereafter asymmetrically coated with a particular coating agent.

Prior to being shipped and sold, the dental wipe of the present invention can be placed in various sealed packaging in order to preserve any additives applied to the dental wipe or otherwise to maintain the dental wipe in a sterile environment. Various packaging materials that can be used include ethylene vinyl alcohol (EVOH) films, film foil laminates, metalized films, multi-layered plastic films, and the like. The packaging can be completely impermeable or can be differentially permeable to the flavorants depending on the application.

The present invention may be better understood by reference to the following example.

As described above, a dental wipe of the present invention can be made from various components and contain various features. For instance, the dental wipe can include a non-elastic component, an elastic component and a moisture barrier. If desired, a texturized surface can be located on the dental wipe for facilitating the scrubbing and cleaning of teeth and gums. Further, the dental wipe can be made from single layer materials or laminates which, in turn, can be made from various materials and fibers. One particular embodiment of a dental wipe made in accordance with the present invention will now be discussed with reference to FIG. 3.

In this embodiment, the dental wipe 10 includes the first section 20 thermally bonded to the second section 30. The second section 30 is designed for contacting the teeth and gums of the user, while the first section 20 is made from an elastic laminate for providing the dental wipe with form fitting properties. More particularly, the second section 30, in this embodiment, is a three layered laminate. The laminate includes an interior polypropylene spunbond layer, a middle moisture barrier layer, and an outer layer that forms an exterior surface of the dental wipe.

The polypropylene spunbond layer is made from spunbond polypropylene filaments and can have a basis weight of from about 0.3 osy to about 1.0 osy, and can particularly have a basis weight of about 0.5 osy. The moisture layer, on the other hand, can be a film made from linear low-density polyethylene containing calcium carbonate filler. The film can be stretched in order to create pores for making the film breathable while remaining substantially impermeable to liquids. The moisture barrier layer can have a basis weight of from about 0.2 osy to about 1.0 osy, and particularly can have a basis weight of about 0.5 osy. The polypropylene spunbond layer can be adhesively secured to the moisture barrier layer.

In an alternative embodiment, the interior polypropylene spunbond layer can be replaced with a nonwoven web made from polypropylene/polyethylene bicomponent fibers. The middle moisture barrier layer, on the other hand, can be a film made from a mixture of polymers, such as CATALLOY film marketed by the Pliant Corporation. The exterior layer can be a spunbond or through air bonded web made from bicomponent polyethylene/polypropylene filaments in a side-by-side arrangement. The exterior layer can have a basis weight of from about 1.0 osy to about 5.0 osy, and can particularly have a basis weight of from about 2.0 osy to about 4.0 osy. Alternatively, the exterior layer itself can be a laminate structure. For example, a two-banked process can be used in which a layer of larger diameter fibers is formed on a layer of small diameter fibers.

The exterior bicomponent spunbond layer can be laminated to the other layers using a thermal or ultrasonic point bonding process, such as a point unbonded pattern process. More particularly, the layers can be point unbonded to form a texturized surface. For instance, as shown in FIG. 3, the point unbonded pattern can be designed to form circular tufts which protrude from the surface of the laminate.

As mentioned above, the first section 20 is an elastic laminate. For instance, the first section 20 can be a stretch bonded laminate sheet. The stretch bonded laminate sheet can include elastic threads made from an elastomeric material sandwiched between two polypropylene spunbond layers. The elastic threads can be, for instance, made from a styrene-ethylene butylene-styrene block copolymer, such as KRATON G2740 available from the Shell Chemical Company. The stretch bonded laminate can have a basis weight of from about 1.0 osy to about 5 osy, particularly from about 1.5 osy to about 3.5 osy, and more particularly from about 2.0 osy to about 3.0 osy.

Instead of a stretch bonded laminate sheet, the first section 20 can also be a neck bonded laminate sheet. The neck bonded laminate sheet can include a metallocene catalyzed elastic polyethylene film sandwiched between two polypropylene spunbond layers. The spunbond layers can have a basis weight of about 0.45 osy prior to being stretched. The polyethylene film, on the other hand, can have a basis weight from about 0.5 osy to about 1.5 osy. The first section 20 can be attached to the second section 30 using various methods. For example, as shown in FIG. 3, the first section 20 can be ultrasonically bonded to the second section 30 along the outer edges in order to form a pocket for the insertion of a finger.

Once the first section 20 and the second section 30 are bonded together, excess material can be cut and removed from the dental wipe. In general, any suitable cutting method can be used in order to trim away excess material. For example, the material can be cut using a high pressure jet of water referred to as a water knife or can be cut using a conventional mechanical device, such as a cutter or a pair of shears. In one embodiment, the first section 20 and the second section 30 can be simultaneously bonded together and cut from the materials from which they are made. For instance, ultrasonic energy can be used to bond and cut materials in one step.

The dimensions of the dental wipe that is formed in accordance with the present invention will depend upon the particular application and purpose for which the dental wipe is to be used. For instance, the dental wipe can be constructed in order to fit around the finger of an adult or the finger of a child. Further, the dental wipe can also be constructed to fit around two fingers. For most single finger dental wipes, the wipe should have a length of from about 1 inch to about 5 inches and a median flattened width of from about 0.5 inches to about 1.5 inches. When constructed to fit around two fingers, the dental wipe can have a median width of from about 0.75 inches to about 2.5 inches, depending on the elasticity of the wipe.

### EXAMPLE

A dental wipe as described above and illustrated in FIGS. 2-4 was coated with a commercially available lip care formulation, specifically BLISTEX® Lip Balm as follows. In addition, section 30 of the dental wipe included an optional mint oil for cleaning and freshening breath. A small amount of lip care formulation was removed from a tube of BLISTEX® Lip Balm by contacting and smearing a finger to the lip care formulation that was extended from the plastic tube the lip balm was supplied in. Lip care formulation was then applied to the dental wipe by contacting the freshly removed lip balm to region 32 illustrated in FIG. 4. The lip care formulation was smeared with a finger onto the wipe in region 32 to form a lip care treatment zone on the dental wipe. The lip care formulation was spread, approximately, in the 2-3 centimeter lower region nearest the open end of the finger-shaped wipe leaving an upper, tooth and gum cleaning region of about 4 centimeters in length. The width of the wipe was about 3 centimeters.

The product was used to clean teeth by placing the finger-shaped wipe over an index finger and then contacting region 33, the oral care treatment zone, to the teeth. Teeth were cleaned by contacting and rubbing region 33 up-and-down and side-to-side over teeth and gum surfaces. The wipe was then withdrawn from the mouth of the user and then region 32, the lip care treatment zone, was contacted to the lower lip of the user. The wipe was rubbed against the lower lip, transferring lip care formulation to the lower lip of the user. The user of the wipe then withdrew the wipe and contacted the lower and upper lips to transfer and spread the lip care formulation more evenly over both lips.

The BLISTEX® Lip Balm that was applied and transferred to the dental wipe included the following listed active ingredients: 2.5 percent by weight oxybenzone and 6.6 percent by weight padimate O sun screening agents and 2 percent by weight dimethicone skin protecting agent. The BLISTEX® Lip Balm also included the following listed ingredients: beeswax, camphor, candelilla wax, cetyl alcohol, cetyl palmitate, cocoa butter, D&C red no. 6, barium lake, lanolin, menthol, methylparaben, mineral oil, ozokerite, paraffic, petrolatum, polybutene, propylparaben and titanium dioxide.

It is understood by one of ordinary skill in the art that the present discussion is a description of exemplary embodiments only, and is not intended as limiting the broader aspects of the present invention, which broader aspects are embodied in the exemplary constructions. The invention is shown by example in the appended claims.

1. A device comprising a hollow member having an open end for insertion of a finger, the hollow member comprising an exterior surface and a lip care formulation disposed on at least a portion of the surface.

2. The device of claim 1, further comprising a texturized surface configured to clean the teeth and gums of a user disposed on the surface.

3. The device of claim 2, wherein the portion of the surface that includes the lip care formulation is separate from the texturized surface or is a portion of the texturized surface.

4. The device of claim 2, wherein the portion of the surface that includes the lip care formulation is a portion of the texturized surface.

5. The device of claim 3, wherein the portion of the surface that includes the lip care formulation is closer to the open end than the texturized surface.

6. The device of claim 2, further comprising a breath freshening agent, a flavoring agent or a dental agent.

7. The device of claim 6, wherein the breath freshening agent, the flavoring agent or the dental agent is disposed on a portion of the surface of the device that is separate from the portion of the surface of the device that includes the lip care formulation.

8. The device of claim 1, wherein the lip care formulation comprises at least one compound selected from the group consisting of allantoin, avobenzone, camphor, dimethicone, homosalate, menthol, meradimate, octinoxate, octisalate, oxybenzone, padimate O, petrolatum, phenol.

9. The device of claim 1, wherein the lip care formulation comprises dimethicone, oxybenzone and padimate O.

10. The device of claim 1, wherein the lip care formulation comprises allantion and camphor, menthol or phenol.

11. The device of claim 1, wherein the lip care formulation comprises dimethicone or petrolatum and at least one sun screening agent.

12. The device of claim 1, wherein the lip care formulation comprises a sun screening agent and a skin protecting agent.

13. The device of claim 1, wherein the lip care formulation comprises an analgesic and a skin protecting agent.

14. The device of claim 1, herein the lip treatment comprises an analgesic selected from the group consisting of camphor, menthol and phenol.

15. The device of claim 1, wherein the lip care formulation comprises a skin protecting agent selected from the group consisting of allantoin, dimethicone and petrolatum.

16. The device of claim 1, wherein the lip care formulation comprises a sun screening agent selected from the group consisting of aminobenzoic acid, anthranilate avobenzone, dioxybenzone, homosalate, lisadimate, menthyl anthranilate, meridamate, octinoxate, octisalate, octocrylene, octyl methoxycinnamate, octyl salicylate, oxybenzone, padimate O, phenylbenzimidazole, roxadimate, sulisobenzone, titanium dioxide, trolamine salicylate and zinc oxide.

17. A disposable substrate for applying a lip treatment composition, the disposable substrate comprising a nonwoven material having a surface upon which is disposed a lip care formulation.

18. The disposable substrate of claim 17 wherein the lip care formulation comprises at least one compound selected from the group consisting of allantoin, avobenzone, camphor, dimethicone, homosalate, menthol, meradimate, octinoxate, octisalate, oxybenzone, padimate O, petrolatum, phenol.

19. The disposable substrate of claim 17 further comprising a breath freshening agent, a flavoring agent or a dental agent.

20. The disposable substrate of claim 17 wherein the lip care formulation comprises a sun screening agent, a skin protecting agent or an analgesic.

21. A packaged disposable product comprising a disposable substrate for applying a lip treatment composition, the disposable substrate comprising a nonwoven material having a surface upon which is disposed a lip care formulation and a package sealed about the disposable substrate.

22. The packaged disposable product of claim 21 wherein the lip care formulation comprises a sun screening agent, a skin protecting agent or an analgesic.
